(19) United States
(12) Patent Application Publication
(10) Pub. No.: US 2006/0193760 A1
(43) Pub. Date: Aug. 31, 2006

(54) Apparatus and method for endoscope soaking basins

(76) Inventor: H. Thomas Hight III, Kennesaw, GA (US)

Correspondence Address: SMITH, GAMBRELL & RUSSELL, LLP, 1230 PEACHTREE STREET, N.E., SUITE 3100, PROMENADE II, ATLANTA, GA, 30309-3592, US

(21) Appl. No.: 11/348,554

(22) Filed: Feb. 6, 2006

Related U.S. Application Data
(63) Continuation-in-part of application No. 10/056,701, filed on Jan. 25, 2002, now Pat. No. 6,994,823.
Application No. 10/056,701 is a continuation-in-part of application No. 09/702,465, filed on Oct. 31, 2000, now Pat. No. 6,361,751.
(60) Provisional application No. 60/296,853, filed on Jun. 9, 2001.

(51) Int. Cl. A61L 2/18

(52) U.S. Cl. 422/292; 422/300

(57) ABSTRACT

The present invention provides an endoscope cleaning apparatus with a circulation system for disinfecting an endoscope. The circulation system includes a pump housing, a pump disposed within the pump housing, and wherein the circulation system pumps the liquid chemical disinfectant through the endoscope disinfecting apparatus to disinfect an endoscope.

### CROSS REFERENCE TO RELATED APPLICATIONS

This application is a continuation-in-part of copending U.S. Utility Application entitled, “Apparatus And Method For Ventilating Endoscope Soaking Basins,” having Ser. No. 10/056,701, filed Jan. 25, 2002, which claims the benefit of U.S. Utility Application entitled, “Apparatus and Method for Disinfecting an Endoscope,” having U.S. Pat. No. 6,361,751, issued Mar. 26, 2002, and U.S. Provisional Application entitled, “Apparatus and Method for Ventilating Endoscope Soaking Basins,” having Ser. No. 60/296,853, filed Jun. 9, 2001, all of which are entirely incorporated herein by reference.

### TECHNICAL FIELD

The present invention is generally related to endoscopes, and more particularly, is related to an apparatus and method for circulating liquid chemical disinfectant in an endoscope cleaning apparatus to efficiently disinfect an endoscope.

### BACKGROUND OF THE INVENTION

Recent improvements in endoscope technology have provided great advances in the practice of medicine. These advances in endoscope technology have enabled medical practitioners to increase the ability to research patient conditions and aid in the diagnosis of heretofore difficult to diagnose conditions. Once the difficult to diagnose conditions are identified, the correct medical treatment can be applied with confidence. In addition to diagnostic applications, endoscopes are used to treat a growing number of conditions.

Endoscopes are widely used on a large variety of organs in the human body, as well as in veterinary applications. These organs include, but are not limited to, digestive, respiratory, urinary, gynecological, orthopedic and otorhinolaryngological systems. The endoscopes used on these various organs of the human body are so expensive that they cannot simply be discarded after a single use on a single patient, but instead must be applied successfully to many patients. Therefore, the used endoscope must be thoroughly cleaned and disinfected before being available for use on another patient.

Currently, there are two types of endoscope cleaning and disinfecting apparatuses and methods. The first is generally utilized in well-financed operations, such as big hospitals. These well-financed institutions generally use an automatic endoscope cleaning and disinfecting machine costing several thousand dollars. The automatic cleaning and disinfecting machine operates much like a dishwasher in cleaning and disinfecting the endoscope. These machines can be costly to operate and repair.

The second type of endoscope cleaning apparatus is a soaking basin. The soaking basin type endoscope cleaning apparatus utilizes a basin to soak an endoscope in a liquid chemical disinfectant. The soaking basins are usually box shaped containers, with lids. The liquid chemical disinfectant is typically glutaraldehyde, a toxic substance, or ortho-phtalaldehyde (i.e. 1,2-benzenedicarboxaldehyde or OPA™). There are numerous problems with using glutaraldehyde. The used and discarded solution is toxic to the environment. The larger the quantity of glutaraldehyde used in the disinfecting process, the more the environmental damage from the discarded solution. Decreasing the quantity of glutaraldehyde needed for this process would benefit the environment.

Moveover, glutaraldehyde and OPA™ produce fumes that can cause health problems for disinfecting workers. The quantity of fumes produced is directly related to the rate of evaporation, which is directly proportional to the size of the liquid surface area where the glutaraldehyde is in contact with the air. The apparatus and method to purposefully diminished the size of the liquid surface area, thereby decreasing the rate of evaporation and the quantity of fumes produced is described in commonly assigned U.S. Pat. No. 6,361,751, issued Mar. 26, 2002, entitled “Apparatus and Method for Disinfecting an Endoscope”, herein incorporated by reference, and in commonly assigned and co-pending U.S. patent application (Attorney Docket 034004-0022) entitled “Apparatus And Method For Ventilating Endoscope Soaking Basins,” having Ser. No. 10/056,701, filed Jan. 25, 2002, herein incorporated by reference.

In order for germ killing to take place, liquid chemical disinfectant needs to circulate across the surface of the endoscope. Currently, soaking basins utilize natural circulation of the glutaraldehyde in the soaking basin. This natural convection occurs as a result of the evaporative process, as warmer liquid chemical disinfectant rises to the surface and is cooled by the evaporative process, the cooler solution moving toward the bottom of the basin. The problem is this: when decrease the rate of evaporation is decreased, the convection currents flowing around the endoscope surface is also decreased, which provides diminished disinfecting capability. Therefore, with a smaller liquid surface area there must a means to circulate the liquid chemical disinfectant, to maximize contact with both the internal and external surfaces of the endoscope.

Another issue with the soaking basin type endoscope cleaning apparatus is the temperature of the liquid chemical disinfectant. In the soaking basin type endoscope cleaning apparatus, the temperature of the glutaraldehyde solution is generally the same as the room temperature or slightly below (i.e. being cooled by the evaporation process). Adequate disinfection is temperature dependent. Longer soaking times are required at room temperature compared to higher temperatures. Therefore, in order to efficiently and adequately disinfect the endoscope, the temperature of the liquid chemical disinfectant needs to be controlled.

Other problems with the disinfecting an endoscope include providing liquid chemical disinfectant into the interior of the endoscope itself. The interior of the endoscope needs to have circulation of liquid chemical disinfectant through it in order to provide sufficient disinfection. Without the circulation of liquid chemical disinfectant through the interior of the endoscope, there is a risk of only partial decontamination.

Thus, a heretofore unaddressed need exists in the industry to address the aforementioned deficiencies and inadequacies.

### SUMMARY OF THE INVENTION

The present invention provides an endoscope cleaning apparatus with a circulation system for disinfecting an endoscope. The circulation system includes a pump housing, a pump disposed within the pump housing, and wherein the circulation system pumps the liquid chemical disinfectant around the endoscope disinfecting apparatus to disinfect an endoscope

Other features and advantages of the present invention will become apparent to one with skill in the art upon examination of the following drawings and detailed description. It is intended that all such additional features and advantages be included herein within the scope of the present invention.

### BRIEF DESCRIPTION OF THE DRAWINGS

The invention can be better understood with reference to the following drawings. The components in the drawings are not necessarily to scale, emphasis instead being placed upon clearly illustrating the principles of the present invention. Moreover, in the drawings, like reference numerals designate corresponding parts throughout the several views.

FIG. 1A is a top view of an endoscope disinfecting apparatus having a coiled insertion tube section and including an embodiment of the circulation system of the present invention.

FIG. 1B is a side view of an endoscope disinfecting apparatus having a coiled insertion tube section and including an embodiment of the circulation system of the present invention.

FIG. 2A is a top view of an alternative embodiment of an endoscope disinfecting apparatus having a coiled insertion tube section and including a heater element for the circulation system of the present invention.

FIG. 2B is a side view of an alternative embodiment of an endoscope disinfecting apparatus having a coiled insertion tube section and including a heater for the circulation system of the present invention, as shown in FIG. 2A.

FIG. 3A is a top view of an alternative embodiment of an endoscope disinfecting apparatus with a variable heating system, that includes the circulation system of the present invention.

FIG. 3B is a side view of an alternative embodiment of an endoscope disinfecting apparatus with a variable heating system, that includes the circulation system of the present invention, as shown in FIG. 3A.

FIG. 4A is a top view of an alternative embodiment of an endoscope disinfecting apparatus having an input probe section and including the circulation system of the present invention.

FIG. 4B is a side view of an alternative embodiment of an endoscope disinfecting apparatus having an input probe section and including the circulation system of the present invention, as shown in FIG. 4A.

FIG. 5A is a top view of an alternative embodiment of an endoscope disinfecting apparatus having an insertion tube section that is offset from the center of the soaking basin, and including the heat pump 100 of the present invention.

FIG. 5B is a side view of the alternative embodiment of an endoscope disinfecting apparatus having an insertion tube section that is offset from the center of the soaking basin, and including the circulation system of the present invention, as shown in FIG. 5A.

FIG. 6A is a top view of an alternative embodiment of an endoscope disinfecting apparatus having an input probe section and including a bidirectional circulation system of the present invention.

FIG. 6B is a side view of an alternative embodiment of an endoscope disinfecting apparatus having an input probe section and including a bidirectional circulation system of the present invention, as shown in FIG. 6A.

### DETAILED DESCRIPTION OF THE PREFERRED EMBODIMENT

The present invention is generally related to endoscopes, and more particularly, is related to an apparatus and method for circulating liquid chemical disinfectant at a controlled temperature in an endoscope cleaning apparatus to efficiently disinfect an endoscope.

Previous endoscope disinfecting basins tend to be tub-shaped and may include a lid to contain vapors from the liquid chemical disinfectant. Because these enclosures rely on natural circulation of liquid chemical disinfectant that normally is at or slightly below room temperature the efficiency of the disinfecting operation is greatly reduced. The soaking time required at room temperature for disinfection is longer, compared to higher temperatures. Embodiments of the present invention seek to maximize the efficiency of the liquid chemical disinfectant.

One method to maximize the efficiency of the liquid chemical disinfectant is to utilize a circulation apparatus in the endoscope disinfecting apparatus. With the diminished evaporation at the liquid surface of this apparatus, which is a safety feature of U.S. Pat. No. 6,361,751, natural circulation of the glutaraldehyde from convection currents formed by surface cooling is decreased. The circulation pump in the endoscope disinfecting apparatus provides circulation of glutaraldehyde in the apparatus, increasing contact between the liquid disinfectant and the endoscope surface.

Another method of increasing or maximizing the efficiency of the liquid chemical disinfectant is to utilize a heater apparatus. The heater would heat the liquid chemical disinfectant to a temperature range that maximizes the efficiency of the disinfecting capabilities. In the example of glutaraldehyde, the solution is most efficient when heated to a temperature greater than 75° F. The heater would also assist in the natural circulation of the glutaraldehyde from convection currents by heating the liquid chemical disinfectant on the bottom of the apparatus.

Still another method of increasing the efficiency of the disinfecting of the endoscope apparatus is to provide liquid chemical disinfectant into the interior of the endoscope itself. This is done by utilizing a coupling on a circulation pump to pump liquid chemical disinfectant through the coupling into the interior of the endoscope apparatus. The circulation of liquid chemical disinfectant through the interior of the endoscope apparatus provides for complete decontamination. The circulation pump may be the same pump providing general circulation through the system, or may be a distinctly separate pump.

Hereinafter, glutaraldehyde, ortho-phtalaldehyde (i.e. 1,2-benzenedicarboxaldehyde or OPA™), or other known medical device disinfectants will be referred to as a liquid chemical disinfectant for the sake of brevity.

Reference will now be made in detail to the description of the invention as illustrated in the drawings. Although the invention will be described in connection with these drawings, there is no intent to limit it to the embodiment or embodiments disclosed therein. On the contrary, the intent is to include all alternatives, modifications, and equivalents included within the scope of the invention as defined by the appended claims.

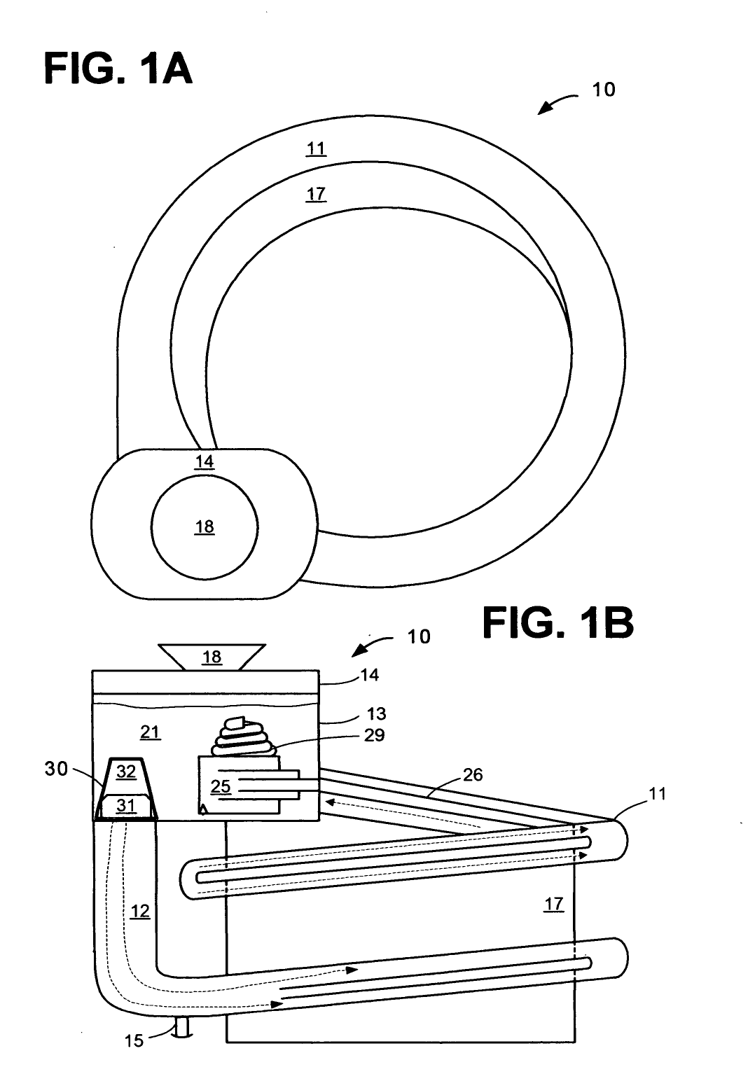

FIG. 1A is a top view of an endoscope disinfecting apparatus 10 having a coiled insertion tube section 11 and including an embodiment of the circulation system 30 of the present invention. As shown, the endoscope disinfecting apparatus 10 includes a soaking basin 13, lid 14, coiled insertion tube section 11, and base 17. The soaking basin 13 includes a volume of liquid chemical disinfectant 21 in which an endoscope 25, insertion tube 26 and air/water and light tube 29, to be disinfected is soaked. During use of the disinfecting apparatus 10, vapors emanate from the surface of the liquid chemical disinfectant 21. In an attempt to minimize the exposure of workers using the disinfecting apparatus 10 to these vapors, a lid 14 and optional fume hood 18 is provided. As such, the vapors from the liquid chemical disinfectant 21 will collect in a vapor space defined by the surface of the liquid chemical disinfectant 21, the walls of the soaking basin 13, the lid 14, and can be removed through flume hood 18.

The coiled endoscope disinfecting apparatus 10 has a soaking basin 13 with lid 14. The outside diameter of the coiled endoscope disinfecting apparatus 10 ranges from a preferred range of 5 to 30 inches. Given one and three quarters coils of insertion tube section 11, as illustrated in FIG. 1A, an endoscope 25 with a 200 centimeter insertion tube 26 would require a diameter of about 17 inches across. A 160 cm insertion tube 26 would require a diameter of about 13 inches across. A 65 cm insertion tube 26 would require a diameter of about 10 inches. While the invention shows the insertion tube section 11 coiling two and three quarters times, it is contemplated by the inventor that the insertion tube section 11 may be coiled through one half to five turns.

From an ease of construction standpoint, the insertion tube section 11 is shown coiled about a support cylinder 17. Note, however, multiple configurations are envisioned that allow the endoscope disinfecting apparatus 10 to rest firmly on a support surface. For example, rather than utilizing a support cylinder 17, the coils of the insertion tube section 1 1 could be separated by support struts (not shown) or simply allowed to rest one on top of another. It is contemplated by the inventor that the enlargement of the soaking basin 13 and the insertion tube section 11 would allow two or more endoscopes to be disinfected at the same time.

FIG. 1B is a side view of an endoscope disinfecting apparatus having a coiled insertion tube section 11 and including an embodiment of the circulation system 30 of the present invention. In the preferred embodiment, the endoscope cleaning apparatus 10 includes circulation system 30 of the present invention. As shown in FIG. 1B, the circulation system 30 is mounted directly in the soaking basin 13. In the example illustrated, the circulation system 30 can pump the liquid chemical disinfectant 21 into returned pipe 12, and expel the liquid chemical disinfectant 21 back into the soaking basin 13. In this manner, the liquid chemical disinfectant 21 can be circulated around the endoscope 25 and insertion tube 26 within the endoscope cleaning apparatus 10. However, it is also contemplated that a reverse circulation pattern is possible. In the reverse circulation pattern, the circulation system 30 would pump the liquid chemical disinfectant 21 from returned pipe 12 into soaking basin 13. In this example, the liquid chemical disinfectant would be drawn from soaking basin 13 around the endoscope 25 and insertion tube 26.

In the preferred embodiment, the circulation system 30 of the present invention comprises a fluid pump 31 and pump housing 32. This fluid pump can be any type of fluid pump, (i.e. magnetic, mechanical, electrical, or hydraulic). In the preferred embodiment, the fluid pump 31 includes, but is not limited to, a magnetically coupled impeller pump. However, it is understood that other types of pumps can be utilized, and the alternative pumps include but are not limited to the following types of pumps, and rotary, gear, vane, piston, centrifugal, propeller, impeller.

The endoscope disinfecting apparatus 10 further includes a draining valve 15 attached at the far end of the insertion tube section 11. The drain valve 15 enables medical staff to quickly and efficiently drain the liquid chemical disinfectant 21 from the endoscope disinfecting apparatus 10. It is also contemplated by the inventor that the drain valve 15 may be comprised of a drain plug or other type of mechanism to allow for controlled flow of the liquid chemical disinfectant 21 from the endoscope disinfecting unit 10. It is also contemplated by the inventor that the drain valve 15 may connect directly to some tube or other liquid containing mechanism to limit evaporation and further prevent medical staff from coming in direct contact with the liquid chemical disinfectant 21.

It is also contemplated by the inventor that sedimentation in the liquid chemical disinfectant 21 may be removed from the endoscope disinfecting apparatus 10 through drain valve 15. Sedimentation refers to small objects in a liquid, such as in this case the liquid chemical disinfectant 21. Sedimentation having a density greater than the liquid chemical disinfectant 21 will have the tendency to collect at the lowest point. In the example shown, the lowest point of the insertion tube 11 is the optimum location of drain valve 15.

Embodiments are also envisioned wherein the fume hood 18 is mounted to either lid 14 (FIG. 1B) or soaking basin 13, and therefore is in fluid communication with the vapor space. In addition, alternative embodiments can include a suction hose (not shown) connecting a fan housing (not shown) to either the lid 14 or soaking basin 13, thereby allowing the fan housing (not shown) to be remotely located from the endoscope disinfecting apparatus 10. Although the fan and motor (not shown) are ideally disposed in separate housings, it is contemplated that both the fan and the motor (not shown) could be stored within a single housing. Moreover, in that the motor is electric, a power cord (not shown) is to be provided.

The insertion tube section 11 is designed in the general shape, size, and length corresponding to the type of endoscope for which the endoscope disinfecting apparatus 10 is specifically designed. The insertion tube section 11 length ranges from a few centimeters to over 350 centimeters long, depending on the length of the endoscope 25 for which the endoscope disinfecting apparatus 10 is specifically designed to disinfect. The diameter of the insertion tube section 11 will range from ¼ inch to the three to four inch range. As shown, the insertion tube section 11 is centered in the bottom of the soaking basin 13. This is ideal if the air/water and light tube (not shown) can be separated from the endoscope 25. However, where the air/water and light tube (not shown) cannot be removed, the insertion tube section 11 can be positioned off center so that the connection between the air/water and light tube (not shown) and the endoscope (not shown) is not unduly stressed by bending.

The endoscope disinfecting apparatus 10 is further comprised of soaking basin 13 that is connected to the insertion tube section 11. The soaking basin 13 supports the body of the endoscope 25. The soaking basin 13 can be configured in a range of shapes and diameters, each shape and diameter designed specifically for the intended endoscope 25. The endoscope soaking basin 13 is specifically designed to reduce the surface area exposure of the liquid chemical disinfectant 21 to reduce the rate of evaporation of the liquid chemical disinfectant 21. The soaking basin 13 can be configured in a wide variety of shapes and sizes corresponding to the type of endoscope 25 for which it is designed. The general diameter of soaking basin 13 can range from 4 inch to 30 inches, but preferably is 4 inches to 15 inches. The soaking basin 13 has a depth of 2 inches to 10 inches, but preferably 4 inches to 8 inches.

To further reduce evaporation of the liquid chemical disinfectant 21, the endoscope disinfecting apparatus 10 further may include a fan hood (not shown) that includes an access door (not shown). This fan hood (not shown) may or may not connect with a water tight/air tight seal to the soaking basin 13. When a water/air tight seal exists, it may be desirable to operate a fan motor (not shown) only when the access door (not shown) is open. This could be accomplished by having an electric switch (not shown) activate the fan motor when the access door is open and deactivate the motor when the access door (not shown) is closed. As well, a ventilation check valve (not shown) could be provided that prevents vapors from escaping from the fan hood (not shown), yet allows adequate inflow to permit operation of the motor. Manual operation of the motor is also envisioned. This fan hood (not shown) further prevents medical staff fume exposure.

Although efforts are made to minimize the surface area of the liquid chemical disinfectant 21 used, vapors will nonetheless be generated and collect inside the fume hood 18. Therefore, an exhaust system (not shown) can be used to remove those fumes that collect in the fume good 18. As previously noted, where a water/air tight seal exists between the feeling hood 18 and the soaking basin 13, intermittent operation of a fan motor (not shown) is probably desired. However, when the seal is not water/air tight, continuous operation of the fan motor (not shown) during use of the endoscope disinfecting apparatus 10 is most desirable in order to minimize the release of vapors.

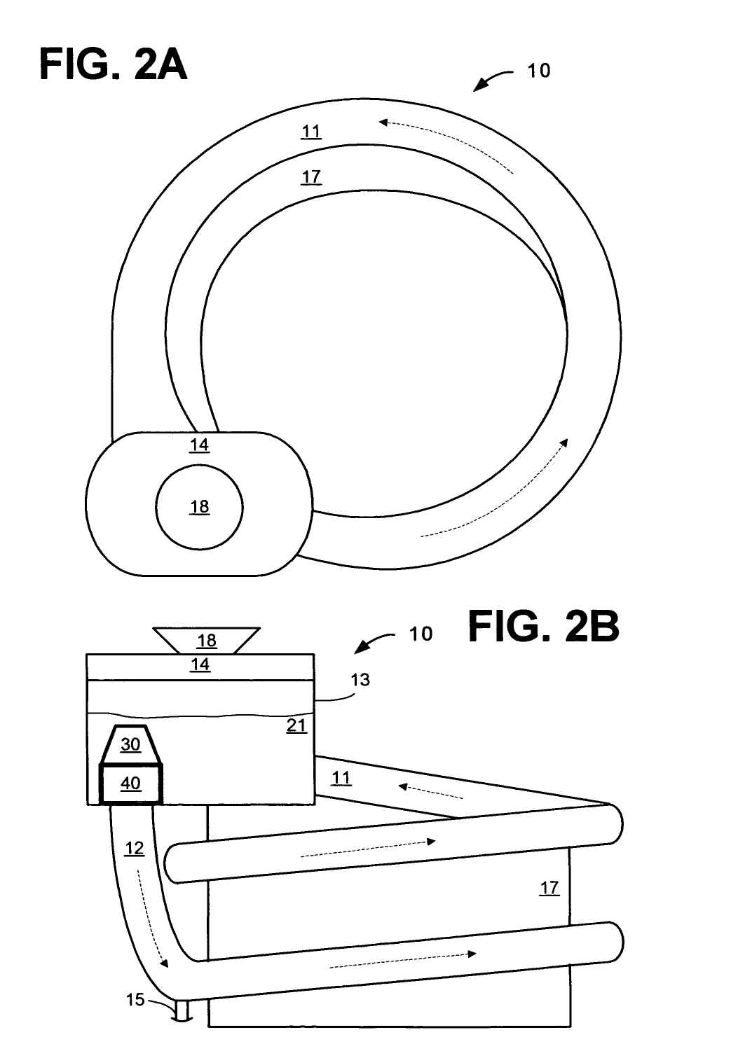

FIGS. 2A and 2B are illustrations of an alternative embodiment of an endoscope disinfecting apparatus 10 having a coiled insertion tube section 11 and including a heater system 40 with the circulation system 30 of the present invention. As discussed before, the endoscope disinfecting apparatus 10 is substantially similar to the endoscope disinfecting apparatus 10 previously discussed with regard to FIG. 1A. However, the distinctions between FIGS. 1A/B and FIGS. 2A/B will be herein discussed in detail. The first distinction of the endoscope disinfecting apparatus 10 is the addition of a heater system 40.

The heater system 40 heats the liquid chemical disinfectant 21 to maximize the efficiency of the disinfecting capabilities. For example, a glutaraldehyde solution is most efficient when heated to a temperature in the range of 75° F. and above. In the alternative embodiment, heater system 40 is provided to increase or maximize the efficiency of the liquid chemical disinfectant 21. Moreover, in that the heater system 40 is normally electric, a power cord (not shown) is to be provided. Other types of heating elements can be utilized for remote undeveloped country locations, such as for example but not limited to, gas, propane, coal, wood or other type of combustion heater element. Consequently, it is understood by the inventor that other types of heating elements may be utilized.

As illustrated in FIGS. 2(A&B), the heater system 40 is used in conjunction with circulation system 30 of the present invention. However, it is contemplated by the inventor in an alternative embodiment that heater system 40 may be used in an endoscope disinfecting apparatus 10 in a stand-alone configuration without the circulation system 30 of the present invention. The heater 40 would assist in the natural circulation of the liquid chemical disinfectant 21 from convection currents by heating the liquid chemical disinfectant 21 on the bottom of the endoscope disinfecting apparatus 10. By utilizing heater 40 the bottom of the endoscope disinfecting apparatus 10, the warmer liquid chemical disinfectant 21 heated by a heater 40 would naturally flow towards the top of the soaking basin 13. For example, a glutaraldehyde solution is most efficient when heated to a temperature in the range of 75° F. and above.

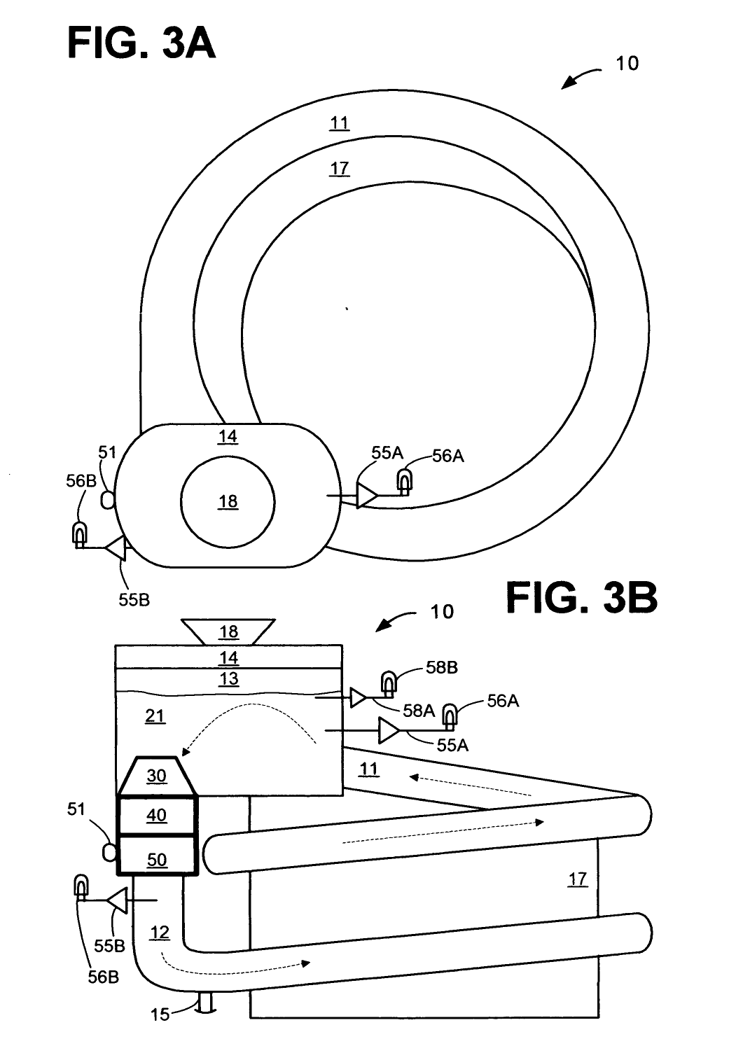

FIGS. 3A and 3B illustrate an alternative embodiment of an endoscope disinfecting apparatus 10 with the circulation system 30 of the present invention that includes a variable heating system 50. As discussed before, the endoscope disinfecting apparatus 10 is substantially similar to the endoscope disinfecting apparatus 10 previously discussed with regard to FIG. 1A. However, the distinctions between FIGS. 1A/B and FIGS. 2A/B will be herein discussed in detail. The first distinction of the endoscope disinfecting apparatus 10 is the addition of a variable heating system 50.

The variable heating system 50 further comprises a temperature control apparatus. The temperature control apparatus further comprises a temperature control switch 51, temperature sensor 55, and a temperature warning light 56. The temperature control switch 51 is connected to the heating element 40 in order to provide a user with the ability to set a minimum temperature for heating the liquid chemical disinfectant 21. In addition to the temperature control switch 51, the temperature control apparatus further in comprises the temperature sensor 55 and temperature warning light 56. The temperature sensor 45 is utilized to measure the temperature of the liquid chemical disinfectant 21 within the endoscope cleaning apparatus 10 of the present invention.

The temperature warning light 56 maybe utilized to indicate that the temperature of the liquid chemical disinfectant 21 is at or above, the indicated temperature on the temperature control switch 51. This would notify a user that the endoscope cleaning apparatus 10 that the liquid chemical disinfectant 21 is at the requested temperature and therefore readily to be utilized to disinfect an endoscope. However, it is also contemplated by the inventor that the temperature warning light 56 maybe utilized just the reverse, which is that the liquid chemical disinfectant 21 is below the indicated temperature on the temperature control switch 51. Therefore, indicating that be liquid chemical disinfectant 21 is not ready to be utilized to disinfect an endoscope.

The variable heating system 50 may be used in a conjunction with multiple temperature sensors 55(a & b) and temperature warning lights 56(a & b) used for indicating when the temperature of the liquid chemical disinfectant 21 is heated to the temperature indicated by the temperature control switch 51. It is contemplated by the inventor is that there are different situations where there are multiple temperature sensors 55 and temperature warning lights 56 would be utilized. One such alternative embodiment would indicate that the minimum temperature selected by temperature control switch 51, was measured by each temperature sensor in the endoscope cleaning apparatus 10.

In another alternative embodiment, a temperature sensor 55 and temperature warning light 56 may be utilized to the measure that the liquid chemical disinfectant 21 meet the minimum temperature, as indicated by temperature control switch 51. In this condition, it makes sense to place the temperature sensor 55a and temperature warning light 56a in the location farthest from heating element 40, as shown in FIG. 3B.

In still another alternative embodiment, a temperature sensor 55 and warning light 56 may be utilized to measure the hottest temperature of the liquid chemical disinfectant 21. In this scenario as illustrated in FIG. 3B, temperature sensor 55b and temperature warning light 56b are immediately downstream from heating element 40. In this configuration, the temperature sensor 55b, and temperature warning light 56b, could indicate when the liquid chemical disinfectant 21 reaches a maximum threshold temperature, thereby indicating that circulation pump 30 has possibly sustained a failure.

In still another alternative embodiment, a surface level sensor 58a and warning light 58b could be included to indicate when the liquid chemical disinfectant 21 has reached a minimum level when the pump 30 or heater element 40 is activated. This prevents damage to either the pump 30 or heater element 40.

As illustrated in FIG. 3B, the circulation pump 30 pushes the liquid chemical disinfectant 21, downward through heater element 40. This flow direction is for illustration purposes only, and it is contemplated by the inventor that the reverse flow is equally possible. If the liquid chemical disinfectant 21 flow is reversed, then the coolest measurement of the liquid chemical disinfectant 21 would be measured at temperature sensor 45b and indicated by temperature warning light 46b. Conversely, in the reverse flow situation, the temperature sensor 45a and temperature warning light 46a would indicate the highest or maximum temperature of the liquid chemical disinfectant 21.

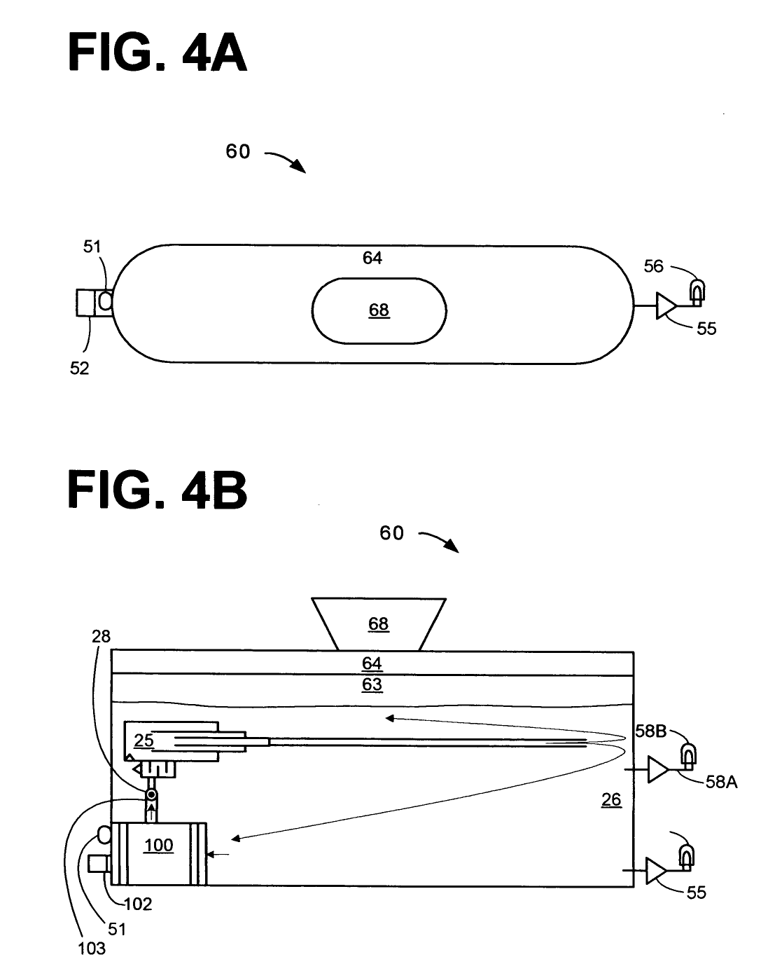

FIGS. 4A and 4B illustrate an alternative embodiment of an endoscope disinfecting apparatus 60 including the heat pump 100 of the present invention and further comprising an input probe connection 103. As discussed before, the endoscope disinfecting apparatus 60 is substantially similar to the endoscope disinfecting apparatus 10 previously discussed with regard to FIG. 1A. However, the distinctions between FIGS. 3A/B and FIGS. 4A/B will be herein discussed in detail.

In this alternative embodiment, the heating system 40 (FIG. 2B) and circulation system 30 (FIG. 2B) are integrated into a heat pump 100. The heating system 40 and circulation system 30 components in the integrated heat pump 100 comprise, but are not limited to the same types of individual components previously discussed above. In this alternative embodiment, a titanium heating element is preferred. However, it is contemplated by the inventor that other types of the heater and pump elements may be utilized.

With the integrated pump 100, the option of a data port 102 is possible. The data port 102 provides the means to download an endoscope disinfection log. The endoscope disinfection log documents a number of different factors considered useful in disinfecting endoscope apparatus 60. These factors include, but are not limited to, the date and time that the endoscope cleaning apparatus 60 is utilized. The endoscope date and time factors led to provide evidence that an endoscope 25 was treated with for at least a minimum allowable time. Other data collected can include the endoscope 25 serial number in order to identify each individual endoscope 25 that is disinfected by the endoscope disinfecting apparatus 60. In addition, it is contemplated that the elapsed time, the minimum and maximum temperature settings and actual minimum and maximum temperatures may be captured and download.

Another feature of this alternative embodiment of endoscope disinfecting apparatus 60 is the addition of an input probe connection 103. The input probe connection 103 is provided in order to channel liquid chemical disinfectant 21 into a port 28 on the endoscope 25. The liquid chemical disinfectant 21 is pumped into the interior of the endoscope 25 in order to provide a more efficient and complete cleaning of the endoscope 25. Since the liquid chemical disinfectant 21 is being channeled through the endoscope 25, it is possible to reduce the interval for disinfecting the endoscope 25, as there is a more efficient cleaning or disinfecting operation that is occurring.

As shown in the FIGS. 4A and 4B, the endoscope cleaning apparatus 60 is an oblong or oval shaped soaking basin 63. The oblong shape is generally utilized for smaller endoscope 25 typed devices, however, it is contemplated by the inventor that larger units may be utilized for endoscopes adding insertion tube sections up to 230 cm. The endoscope cleaning apparatus 60 includes cover 64 and vent 68 that are described hereinabove in further detail.

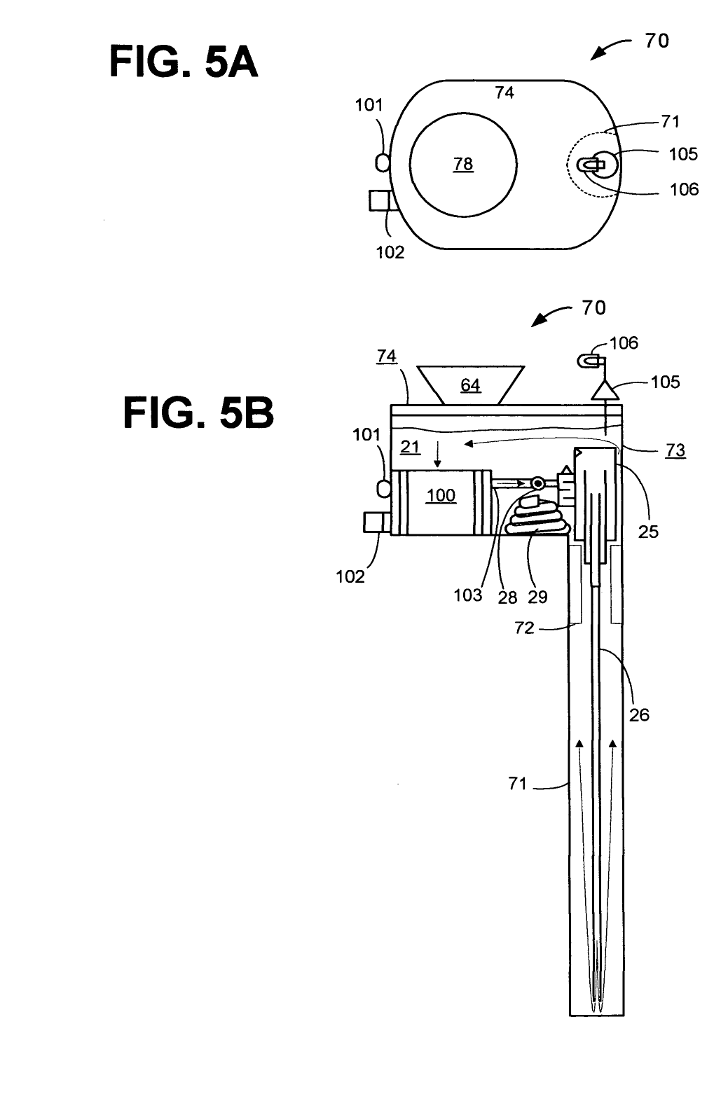

FIGS. 5A and 5B are illustrations of an alternative embodiment of an endoscope disinfecting apparatus 70 having an endoscope insertion tube section 71 that is offset from the center of the soaking basin 73 and including the heat pump 100 of the present invention. Having the insertion tube section 71 offset from the center of the soaking basin 73 enables the endoscope disinfecting apparatus 70 to be placed on a countertop with the insertion tube section 71 hanging over the edge of the counter towards a floor.

In this alternative embodiment, the counter space required for the endoscope disinfecting apparatus 70 is minimized, as well as the surface area of the liquid chemical disinfectant 21. The insertion tube section 71 length ranges from a few centimeters to over 350 centimeters long, depending on the length of the endoscope 25 for which the endoscope disinfecting apparatus 10 is specifically designed to disinfect. In this alternative embodiment, the insertion tube section 71 preferably is at least 80 centimeters long.

As seen in FIG. 5B, it is even more clearly illustrated that the air/water and light tube 29 is contained within a liquid chemical disinfectant 21, whereby the connection of the air/water light tube 29, insertion tube 26 and the endoscope 25 is not stressed. It is also depicted in FIG. 5B that the endoscope insertion tube section 61 is offset from the center of the soaking basin 69. This offset is further implemented to relieve any stress on the connection of the insertion tube 26 with the endoscope 25. This is further shown in the top view of FIG. 5A wherein the endoscope 25 is inserted in the endoscope disinfecting apparatus 60 and offset from the center of the soaking basin 73.

In this alternative embodiment, the heating system 40 (FIG. 2B) and circulation system 30 (FIG. 2B) are integrated into a heat pump 100. The heating system 40 and circulation system 30 components in the integrated heat pump 100 comprise, but are not limited to the same types of individual components previously discussed above.

As shown, the heat pump 100 comprises a variable heating system including a temperature control switch 101, data port 102, temperature sensor 105, and a temperature warning light 106. These components operate as described above, with regard to FIGS. 4A and 4B. Also included in heat pump 100 is input probe connection 103. This input probe connection operates as discussed above with FIGS. 4A and 4B.

In the alternative embodiment illustrated in FIG. 5B, the input probe 103 pumps heated liquid chemical disinfectant 21 into port 28 of the endoscope 25. This heated liquid chemical disinfectant 21 is then pumped throughout the endoscope 25, including insertion tube 26 that extends into offset endoscope insertion tube section 71. This provides for quicker and more thorough disinfection of endoscope 25.

It is also contemplated by the inventor, that input probe 103 contain a valve that provides the pulsing of the liquid chemical disinfectant 21 through the port 28. The pulsing of the liquid chemical disinfectant 21 provides for efficient cleaning. It is also contemplated that the input probe 103 would provide a loose connection to port 28, in order to allow liquid chemical disinfectant 21 to flow over the surface of port 28.

As shown, endoscope 25 is supported by insertion tube support blocks 72. The insertion tube support blocks 72 prevent endoscope 25 from the descending too deeply into the offset endoscope insertion tube section 71, which could cause damage to insertion tube 26. The endoscope cleaning apparatus 70 includes cover 74 and vent 78 that are described hereinabove in further detail in other embodiments.

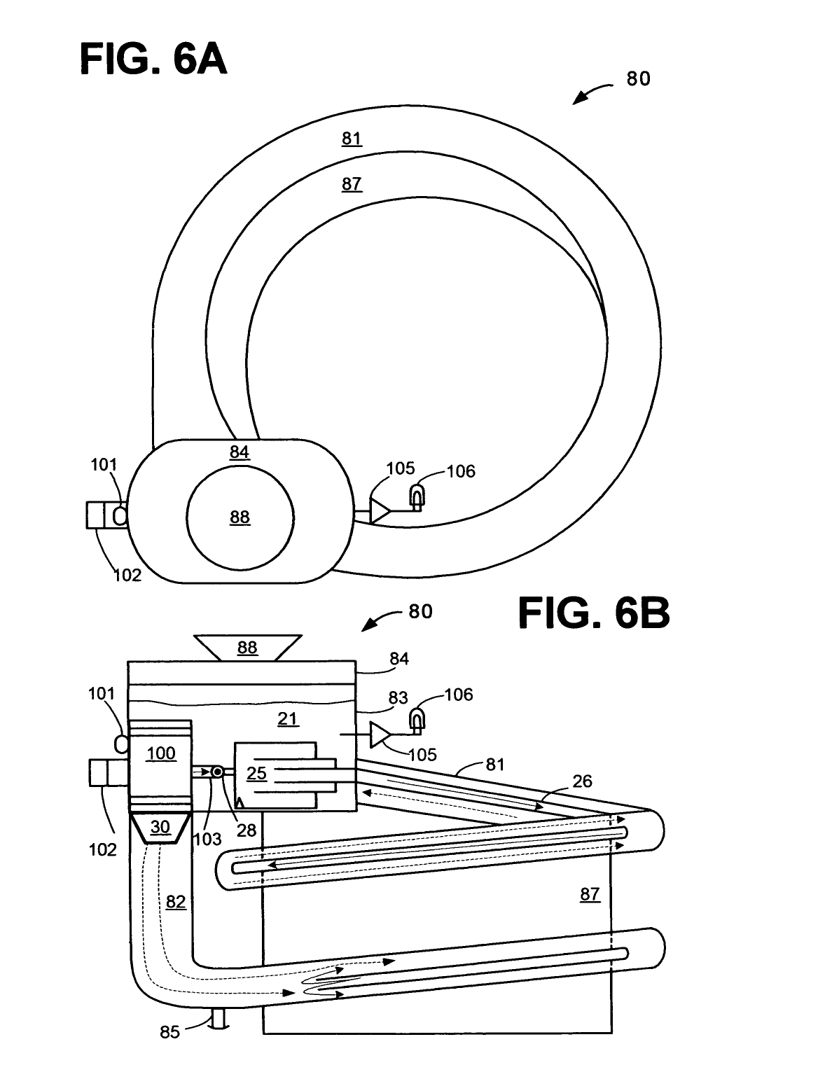

FIG. 6A is a top view of an alternative embodiment of an endoscope disinfecting apparatus 80 that provides for bidirectional circulation of the liquid chemical disinfectant 21 in the endoscope disinfecting apparatus 80 present invention. The bidirectional circulation provides for more efficient cleaning of the endoscope 25. The bidirectional circulation also provides for the ability to use different temperatures on different parts of the endoscope 25.

As shown in FIG. 6A, endoscope disinfecting apparatus 80 includes soaking basin 89, lid 88, coiled insertion tube section 81 and base 87. Also illustrated is temperature control switch 101, temperature sensor 105, temperature warning light 106 and data port 102, which are herein defined in further detail above with regard to FIGS. 3A and 4A.

FIG. 6B is a side view of an alternative embodiment of an endoscope disinfecting apparatus 80 that provides for bidirectional circulation of liquid chemical disinfectant 21 of the present invention, as shown in FIG. 6A. Illustrated in the side view is heat pump 100 utilizing input probe connection 103 to pump liquid chemical disinfectant 21 into endoscope 25 through port 28. In this configuration in the liquid chemical disinfectant 21 is heated to a minimum temperature set by temperature control switch 101. The liquid chemical disinfectant 21 is then pumped through the endoscope 27 in a direction opposite of the liquid chemical disinfectant 21 that is pumped through the disinfecting apparatus 80 by pump 30. In this manner the heated liquid chemical disinfectant 21 from heat pump 100 is pumped in a direction opposite of the liquid chemical disinfectant 21 that is pumped directly through the endoscope 25. In this manner, it is possible than to have a bidirectional flow, which provides for superior disinfection.

It should be understood that heat pump 100 can be designed to provide for the bidirectional circulation of liquid chemical disinfectant 21 without an extra pump. This would be accomplished by having heat pump 100 with a separate flow tube (not shown) from the main pump that provides pumped liquid disinfectant 21 into input probe connection 103. In this alternative embodiment, only one pump is needed to produce the directional circulation.

Conversely, it should also be understood that multiple pumps and heaters can be provided to provide for bidirectional circulation of liquid chemical disinfectant in the endoscope cleaning apparatus 80, of the present invention. In this alternative embodiment, additional pumps (not shown) and heaters (not shown) can be provided for multiple circulation pathways. In a multiple pump and heater environment, a separate pump and heater can be provided for circulation in endoscope cleaning apparatus, 80, the endoscope port 28 and air/water and light tube 29. Multiple pumps and heaters can also be provided to pump liquid chemical disinfectant 21 through multiple ports on endoscope 25.

In addition, it is also understood that multiple pumps and heaters can be provided to the disinfectant multiple endoscopes 25.

It should be emphasized tube the above-described embodiments of the present invention, particularly, any “preferred” embodiments, are merely possible examples of implementations, merely set forth for a clear understanding of the principles of the invention. Many variations and modifications may be made to the above-described embodiment(s) of the invention without departing substantially from the spirit and principles of the invention. All such modifications and variations are intended to be included herein within the scope of the present invention and protected by the following claims.

Therefore, having thus described the invention, at least the following is claimed:

1. An endoscope cleaning apparatus with a circulation system for disinfecting an endoscope, said circulation system comprising:
a pump housing;
a pump disposed within said pump housing; and
wherein said pump housing is connected to the endoscope disinfecting apparatus to circulate the liquid chemical disinfectant in the endoscope cleaning apparatus to disinfect an endoscope.

2. The system of claim 1, wherein said endoscope disinfecting apparatus further includes
a soaking basin, wherein said pump housing is connected to said soaking basin; and
a basin lid, wherein the basin lid is configured to contain fumes from the liquid chemical disinfectant within a vapor space defined by said lid said soaking basin, a surface of the liquid chemical disinfectant in said soaking basin.

3. The system of claim 2, further comprising a second pump that connects to the endoscope to pump liquid chemical disinfectant though the interior of the endoscope.

4. The system of claim 1, wherein said endoscope cleaning apparatus further includes a heater element for heating the liquid chemical disinfectant.

5. The system of claim 4, wherein said heater element is titanium.

6. The system of claim 5, wherein said endoscope cleaning apparatus further includes a temperature control switch that that sets the minimum temperature for heating the liquid chemical disinfectant.

7. The system of claim 6, wherein said endoscope cleaning apparatus further includes a temperature sensor for measuring the temperature of the liquid chemical disinfectant.

8. The system of claim 7, wherein said endoscope cleaning apparatus further includes a temperature warning element for indicating that the temperature of the liquid chemical disinfectant has exceeded a preset maximum.

9. The system of claim 7, wherein said endoscope cleaning apparatus further includes a temperature warning element for indicating that the temperature of the liquid chemical disinfectant is below the temperature set by the temperature control switch.

10. The system of claim 7, wherein said endoscope cleaning apparatus further includes a temperature warning element for indicating that the temperature of the liquid chemical disinfectant has obtained the temperature set by the temperature control switch.

11. An endoscope cleaning apparatus with a heating system for disinfecting an endoscope, said heating system comprising:
a housing;
a heating element disposed within said housing; and
wherein said housing is connected to the endoscope disinfecting apparatus to heat the liquid chemical disinfectant in the endoscope disinfecting apparatus to disinfect an endoscope.

12. The system of claim 11, wherein said endoscope cleaning apparatus further includes a temperature warning element for indicating that the temperature of the liquid chemical disinfectant has exceeded a preset maximum.

13. The system of claim 11, wherein said endoscope cleaning apparatus further includes a temperature control switch that that sets the minimum temperature for heating the liquid chemical disinfectant.

14. The system of claim 13, wherein said endoscope cleaning apparatus further includes a temperature sensor for measuring the temperature of the liquid chemical disinfectant.

15. The system of claim 14, wherein said endoscope cleaning apparatus further includes a temperature warning element for indicating that the temperature of the liquid chemical disinfectant is below the temperature set by the temperature control switch.

16. The system of claim 14, wherein said endoscope cleaning apparatus further includes a temperature warning element for indicating that the temperature of the liquid chemical disinfectant has obtained the temperature set by the temperature control switch.

17. The system of claim 11, wherein said endoscope disinfecting apparatus further comprises:
a disinfecting basin that disinfects a body, eye piece, an air/water and light tube of the endoscope; and
an insertion tube section connected to said endoscope basin that disinfects an insertion tube of the endoscope.

18. The system of claim 17, further comprising a pump that connected to the endoscope disinfecting apparatus to circulate the liquid chemical disinfectant in the endoscope cleaning apparatus to disinfect the endoscope.

19. The system of claim 17, further comprising a second pump that connects to the endoscope to pump liquid chemical disinfectant though the interior of the endoscope.

20. The system of claim 18, wherein said pump circulates the liquid chemical disinfectant around the endoscope in a direction opposite of the liquid chemical disinfectant pumped through the interior of the endoscope.
